(19) United States
(12) Patent Application Publication
(10) Pub. No.: US 2004/0171900 A1
(43) Pub. Date: Sep. 2, 2004

(54) Method and device for reactions start-up

(75) Inventors: Daxiang Wang, Ponca City, OK (US); Chad Ricketson, Ponca City, OK (US); Gloria I. Straguzzi, Ponca City, OK (US); Harold A. Wright, Ponca City, OK (US); Larry D. Swinney, Stillwater, OK (US); Joe D. Allison, Ponca City, OK (US); Zhen Chen, Ponca City, OK (US); Kevin L. Ricketson, Ponca City, OK (US); Shang Y. Chen, Oklahoma City, OK (US); Steven R. McDonald, Ponca City, OK (US)

Correspondence Address: DAVID W. WESTPHAL, CONOCOPHILLIPS COMPANY, P.O. BOX 1267, PONCA CITY, OK, 74602-1267, US

(73) Assignee: ConocoPhillips Company, Houston, TX

(21) Appl. No.: 10/377,356

(22) Filed: Feb. 28, 2003

(51) Int. Cl. C07C 5/32; C07C 2/00; B01J 8/02

(52) U.S. Cl. 585/658; 585/943; 422/198; 422/211

(57) ABSTRACT

The present invention includes methods and apparatus for start-up a chemical reactor wherein at least a portion of the igniter is downstream from the reaction zone which needs to be ignited. Particularly, embodiments of the present invention include a partial oxidation reactor with an igniter downstream of the partial oxidation zone.

### CROSS REFERENCE TO RELATED APPLICATIONS

Not applicable.

### STATEMENT REGARDING FEDERALLY SPONSORED RESEARCH

Not applicable.

### FIELD OF THE INVENTION

The current invention relates to a system for starting-up or initiating a thermally self-sustaining chemical reaction.

### BACKGROUND

A significant amount of natural gas is situated in areas that are geographically remote from population and industrial centers. The costs of compression, transportation, and storage may make its use economically unattractive. To help improve the economics of natural gas use, much research has focused on the use of methane, the main component of natural gas, and other light hydrocarbons (e.g., C₁-C₄) as a starting material for the production of hydrocarbon liquids (e.g., C₅₊), which may be more easily transported and thus more economical. One method of converting light hydrocarbons to heavier hydrocarbons comprises partially oxidizing at least a portion of the light hydrocarbons to a mixture of CO and H₂ (i.e., syngas or synthesis gas) and further converting the syngas to higher hydrocarbons in a hydrocarbon synthesis reactor (e.g., Fischer-Tropsch).

In some catalytic partial oxidation processes, a hydrocarbon feedstock may be preheated and mixed with an oxygen source, such as air, oxygen-enriched air, or oxygen, and introduced to a catalyst at elevated temperature and pressure. The syngas in turn may be converted to hydrocarbon products, for example, fuels boiling in the middle distillate range, such as kerosene and diesel fuel, and hydrocarbon waxes by hydrocarbon synthesis process, such as, by way of example only, the Fischer-Tropsch Synthesis. An example of Fischer-Tropsch synthesis is disclosed in U.S. Pat. No. 6,365,544 to Herron et al., incorporated herein by reference.

hi order to initiate a catalytic partial oxidation (CPOX) process, it may be necessary to preheat the catalyst to a temperature at which ignition (i.e., initiation) of the partial oxidation reaction occurs. This may be problematic because, inter alia, catalytic partial oxidation reactors are typically very small, and providing an ignition source for catalyst heating can complicate the process and significantly add to the size and cost of the syngas reactor system. For example, the use of a preheating torch or burner, may not be practical for catalytic partial oxidation processes, and too rapid heating of the catalyst bed may destroy the catalyst due to thermal stress. A feed comprising H₂, O₂, and a diluent (e.g., nitrogen, helium, argon, steam, methane, CO, CO₂, ethane, propane, butane, alcohols, or olefins) may be employed to ignite the catalyst bed and control heat-up.

Another technique for initiating a CPOX process may be preheating the feed upstream of the catalyst up to 500° C., or more. However, this practice may present an increased safety hazard as well as an increased risk of cracking the hydrocarbon feed during preheat. Additionally, preheat equipment may increase the capital cost of a syngas production unit.

Another method for igniting a CPOX reactor is to briefly spike or supplement the feed with an “ignition agent,” which is typically a partially oxidizable gas that is more readily oxidizable than the partial oxidation hydrocarbon feed to be partially oxidized. For example, to keep the preheat temperature below about 500° C., up to about 50% by volume of propane may need to be spiked into the hydrocarbon feed to initiate the CPOX reaction. Ammonia may also be an ignition agent. After the catalyst reaches a temperature of about 1000° C., the ignition agent may be removed. Use of an ignition agent (e.g., propane or ammonia) not only complicates the syngas production procedure, but there are additional costs associated with handling of the ignition agent, additional safety considerations, and possible detrimental effects on the efficiency of the syngas system due to possible coke deposition.

Another method of startup may be lighting off a reactor using an upstream catalytic burner. Hydrocarbon fuel and an oxidizer are fed to a highly active oxidative catalyst to initiate a combustion reaction and the heat released from the combustion heats up the downstream partial oxidation catalyst zone. After ignition, the combustion reaction in the ignition zone is quenched using steam. However, this method may cause a loss of CO and H₂ selectivity due to the ignition catalyst catalyzing a reaction other than the desired selective oxidation prior to the reactants contacting the downstream partial oxidation catalyst.

Thus, it would be desirable to have an ignition system which minimizes the loss of selectivity, loss of production of CO and H₂, and premature deactivation of the catalyst and which requires minimal (if any) additional handling equipment or cost.

### SUMMARY

Embodiments of the present invention comprise partial oxidation chemical reactors which have an ignition or light-off system downstream of the reaction zone. In other embodiments, the downstream ignition system ignites and initiates a sulfur partial oxidation reaction (such as that described in Published U.S. patent application Ser. Nos. 20020134706 or 20020131928 to Keller et al., both incorporated herein by reference) or an oxidative dehydrogenation reaction or catalytic partial oxidation of a hydrocarbon gas in a reaction zone to produce synthesis gas.

### BRIEF DESCRIPTION OF THE DRAWINGS

FIG. 1 is a schematic drawing of embodiments of the present invention.

FIG. 2 is a schematic drawing of a laboratory scale reactor in accordance with embodiments of the present invention.

FIG. 3 is a graph of a rapid temperature increase expected at start-up of a thermally self-sustaining reaction.

### DETAILED DESCRIPTION

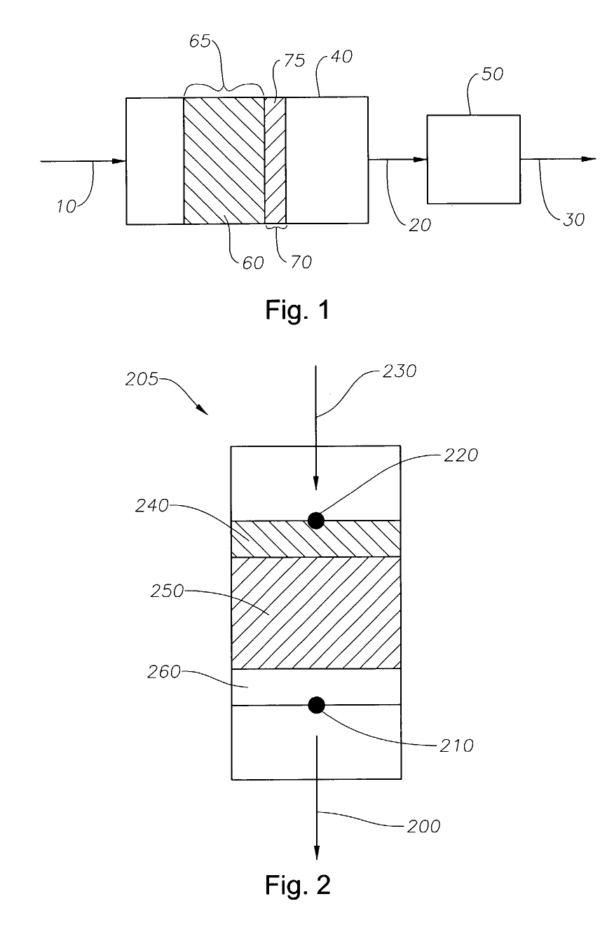

There is shown in FIG. 1, a schematic drawing of a reaction system comprising a feed stream 10, a syngas reactor 40, a reaction zone 65, an ignition zone 70, a syngas stream 20, a hydrocarbon synthesis reactor (e.g., Fischer-Tropsch) reactor 50, and a product stream 30.

In the reaction system of FIG. 1, preheated feed stream as methane and an oxygen-containing gas, such as substantially pure oxygen is mixed and introduced into syngas reactor 40. The feed stream 10 is charged into the reactor 40 and initially proceeds through reaction zone 65 and ignition zone 70. Reaction zone 65 may be charged with syngas catalyst 60 and ignition zone 70 may be charged with an igniter such as an ignition catalyst 75. An “igniter” is any chemical means for igniting at least a portion of the feed stream. Upon its initial impingement with an ignition catalyst 75 in ignition zone 70, the feed stream ignites and produces substantial amounts of heat. At least a portion of the heat then transfers to reaction zone 65 creating conditions favorable to partially oxidize the hydrocarbon in the feed stream as it flows through reaction zone 65. As reaction zone 65 approaches reaction conditions, the heat generated by the partial oxidation reaction will be enough to sustain the reaction in the reaction zone 65. Additionally, as the reaction zone 65, approaches reaction conditions, the oxygen from the feed stream 10 is preferably substantially expended. Thus, the non-selective reactions in the ignition zone 70 may subside because there is a lack of oxidant. Accordingly, opportunity for the ignition zone 70 to decrease the overall selectivity of the reactor 40 or to decrease the production of the desirable products (i.e., CO and H₂) may subside. Therefore, after ignition and light-off of the reaction in the reaction zone 65, the presence of the ignition zone 70 does not substantially adversely effect the performance of the reactor 40.

Reaction zone 65 preferably, but not necessarily, contains a catalyst for catalyzing the partial oxidation reaction, such as supported rhodium and samarium. The catalyst may comprise any acceptable syngas catalyst, such as, for example nickel, ruthenium, palladium, osmium, iridium, samarium, cobalt, platinum, rhodium, Ni—MgO, Group VIII metals, mixed oxides (e.g., perovskites), or combinations thereof. Preferably, the support comprises substantially spherical alumina or zirconia particles having a diameter of about 1 mm. The support may also be any other suitable support, such as, for example, pellets, pills, foams, monoliths, beads, particulates, granules, rings, ceramic honeycomb structures, wire gauze or any other suitable supports in any acceptable shape. Likewise, the support may be made of any acceptable refractory material (pure, modified, or doped) such as, for example, titania, silica, zirconia, alumina, zinc oxide, phosphates (such as aluminum phosphates or silica alumina phosphates), partially stabilized zirconia, or other refractory oxides or mixtures thereof. It is also envisioned that it is not always necessary that the catalyst be supported. For example, an unsupported catalyst may be in the form of wire gauze, wire mesh, metal shot, or a metal monolith. There are a plethora of catalyst systems which would be acceptable and are contemplated to fall within the scope of the present invention, such as those disclosed in STRUCTURED CATALYSTS AND REACTORS, 179-208, 599-615 (Andrzej Cybulski and Jacob A. Moulijn eds. 1998) and Published U.S. patent application Ser. No. 20020115730 to Allison et al., both incorporated herein by reference.

In reaction zone 65, the hydrocarbon in the feedstream is partially oxidized to syngas stream 20 which comprises primarily CO and H₂. The syngas stream 20 is cooled, recovered, and treated before introduction into hydrocarbon synthesis (e.g., Fischer-Tropsch) reactor 50. Typically, hydrocarbon synthesis reactor 50 contains a catalyst which comprises one or more metals such as Group VIII metals such as iron, nickel, or cobalt and one or more other metals such as rhenium, ruthenium, thorium, zirconium, hafnium, uranium, or lanthanum, all may be supported on a refractory metal oxide such as those mentioned above. In the hydrocarbon synthesis reactor, the syngas stream reacts to form product stream 30 which generally comprises liquid hydrocarbons. The product stream 30 may be manipulated by manipulating the conditions in reactor 50:

Ignition catalyst 75 in ignition zone in embodiments of the present invention comprises using novel, highly active metal oxide supported metal/metal oxide catalysts to initiate the exothermic reaction. The ignition catalyst 75 comprises a metal A, preferably comprises a metal A and a metal C, more preferably comprises a metal A, a metal B, and a metal C, wherein:

A is one of the precious metals Rh, Ru, Pd, Pt, Au, Ag, Os or Ir or is a transition metal chosen from the group consisting of Sc, Ti, V, Cr, Mn, Fe, Co, Ni, Cu, Zn, Nb, Mo, Tc, Ru, Rh, Pd, Ag, Hf, Ta, W, Re, preferably Pt, Pd, Au, Ag, Fe, Co, Ni, Mn, V or Mo or any combination of any of the above;

B is a rare earth metal La, Ce, Pr, Nd, Pm, Sm, Eu, Gd, Th, Dy, Ho, Er, Tm, Yb, Lu, Sc, Y and Th, preferably La, Yb, Sm or Ce;

C is an element chosen from Mg, Ca, Sr, Ba, Ra, Al, Ga, In, Tl, Ti, Zr, Si, Ge, Sn, and Pb preferably Mg, Al, Ti, Zr or Si.

These catalysts can have the general formula αAOₓ−βBO_{y}−γCO_{z}, wherein:

O is oxygen;

α, β,γ are the relative molar ratios of each metal oxide and preferably α=0.0001−0.2; β=0−0.5; γ=0.5−1; and

x, y, z are the numbers determined by the valence requirements of the metals A, B, and C, respectively. Their value can be zero when the corresponding metal remains in the metallic state.

By way of example only, in this general formula, if component A is in metallic form, this general formula can be presented as αA−βBO_{y}−γCO_{z}. Alternatively, the catalyst can take a general formula as αAOₓ−γCO_{z}, when component B is not used. The codes A, C, O, α, γ, x, z, etc. have the same meaning as described above. Furthermore, if component A is in metallic form, this general formula becomes αA−γCO_{z}.

Test Procedure

The catalysts were evaluated for their ability to ignite the partial oxidation reaction in a conventional flow apparatus using a quartz reactor with a length of 12 inches, an outside diameter of 19 mm and an inside diameter of 13 mm. Ceramic foam pieces of 99% alumina (½″ outside diameter×⅝″ thick, with 80 pores per linear inch) were placed before the syngas reaction catalyst as radiation shields. The inlet radiation shield also aided in uniform distribution of the feed gases. Either a blank 80 ppi alumina monolith or one of the ignition catalysts prepared in examples 1 and 2 was loaded after the syngas catalyst bed.

An Inconel®-sheathed, multi-point K-type (Chromel/Alumel) thermocouple was placed axially inside the reactor, touching the top (inlet) face of the radiation shield. The temperature of the point touching the leading edge of the shield is defined as the inlet temperature. Another Inconel®-sheathed, multi-point K-type thermocouple was positioned axially touching the bottom radiation shield, and was used to indicate the exit temperatures. The temperature of the point touching the bottom edge of the lower shield is defined as the exit temperature. The multi-point thermocouples provide preheating and quenching profiles in the reactor system.

For catalyst light-off, different procedures can be used. One light-off procedure may consist of placing a combustion catalyst-doped bottom radiation shield beneath the catalyst bed. The ignition catalyst(s) and the radiation shields were tightly sealed against the inside walls of the quartz reactor by wrapping the pieces radially with a high purity (99.5%) alumina paper. A 600-watt band heater set at 90% electrical output was placed around the quartz tube just about the top radiation shield, providing heat to light-off the reaction and preheat the feed gases.

Oxygen was mixed with the methane through a static mixer immediately before the mixture entered the catalyst system. Typical light-off oxygen to methane volumetric ratios range from 0.1 to 0.6.

The reactor was heated up stepwise at 5° C. per step in gas mixture containing 2258 ml/min methane and 1242 ml/min oxygen. The observed indication of light-off of the selective oxidation reaction was defined by the exit temperature increasing quickly and rising well above the inlet gas temperature.

When propane was used as spike gas to help the ignition, the reactor was heated in a gas mixture containing 1000 ml/min methane, 1000 ml/min propane, and 400 ml/min oxygen. After ignition the propane was immediately removed.

The reaction gases were cooled through ambient cooling followed by a cooling coil wrapped around the bottom section of the quartz tube. The cooling coil provided sufficient cooling to protect the viton rings used in sealing the bottom edge of the quartz reactor.

After light-off, the feed mixture was adjusted to a total flow of 5000 cc/min with an oxygen/methane molar ratio of 0.55. Reaction pressure was less than 5 psig (136 kPa). The reactor effluent was analyzed using a gas chromatograph equipped with a thermal conductivity detector. Catalytic performance was calculated according to the GC data.

$${CH}_{4} conversion ( \% ) \text{:} X ( {CH}_{4} ) = \frac{{[ {CH}_{4} ]}_{i n} - {[ {CH}_{4} ]}_{out}}{{[ {CH}_{4} ]}_{i n}} \times 100 \%$$

$$CO selectivity ( \% ) \text{:} S ( CO ) = \frac{{[ CO ]}_{produced}}{{[ {CH}_{4} ]}_{i n} - {[ {CH}_{4} ]}_{out}} \times 100 \% ;$$

$$Hydrogen selectively ( \% ) \text{:} S ( {H}_{2} ) = \frac{{0.5 [ {H}_{2} ]}_{produced}}{{[ {CH}_{4} ]}_{i n} - {[ {CH}_{4} ]}_{out}} \times 100 \% ;$$

wherein [CH₄]=methane molar flow, [H₂]=hydrogen molar flow, and [CO]=CO molar flow.

General Procedure for Igniting Catalyst Preparation

Embodiments of catalysts which may be used in embodiments of the present invention can be prepared through any impregnation or co-precipitation techniques known in the art. Impregnation techniques are more preferred, especially when noble metals such a Pt and/or Au are being used.

When the catalysts are prepared by impregnation, first, a support material must be selected. It is preferred that the support material have a high surface area and a wide variety of pore structures. Although many support materials are suitable, the preferred support material would be selected from the group comprising alumina, silica, titania, magnesia, zirconia, silicon carbide, active carbon and mixture thereof After selecting a support material, a liquid solution containing the active metal components is impregnated onto the support using either incipient wetness or by soaking the support in excess solution. The solid material is then dried starting at room temperature and then ramped up to around 120° C. The resulting catalyst material is then calcined at 200° to 800° C. to decompose the precursor compound(s) into their corresponding metal oxides.

When multi-components are used, such as expressed in the formula of αAOₓ−βBO_{y}−γCO_{z}, stepwise or co-impregnation can be used. Stepwise impregnation is done by impregnating one component, as described above, followed by the impregnation of the next component. Calcination in between the impregnation of each component is optional depending on the exact metals used. Alternatively, a co-impregnation method can be used in preparing multi-components catalysts. In this method, a mixed solution containing all desired metal elements is impregnated onto the catalyst support material in one step followed by drying and calcination.

Some of the preferred catalysts may be active after calcination. However, most catalysts may need to be reduced after calcination to achieve an active catalyst. The calcined catalyst are usually reduced in a gas mixture containing hydrogen in the temperature range of 200°-700° C. to convert the active component from oxide to its metallic state.

The following examples and descriptions are intended to illustrate but not limit the present invention.

Process of Producing Syngas

A feed stream comprising a light hydrocarbon feedstock and an O₂-containing gas is contacted with a controlled pore structure catalyst that is active for catalyzing the conversion of methane or natural gas and molecular oxygen to Primarily CO and H₂ by a net catalytic partial oxidation (CPOX) reaction. Preferably a very fast contact (i.e., milliseconds range)/fast quench (i.e., less than one second) reactor assembly is employed. Several schemes for carrying out catalytic partial oxidation (CPOX) of hydrocarbons in a short contact time reactor are well known and have been described in the literature. For example, U.S. Pat. No. 6,488,907 to Barnes et al. and U.S. Pat. No. 6,409,940 to Gaffney et al., both incorporated herein by reference. The light hydrocarbon feedstock may be any gaseous hydrocarbon having a low boiling point, such as methane, natural gas, associated gas, or other sources of C₁-C₅ hydrocarbons. The hydrocarbon feedstock may be a gas arising from naturally occurring reserves of methane which contain carbon dioxide. Preferable, the feed comprises at least 50% by volume methane, more preferably at least 75% by volume, and most preferably at least 80% by volume methane. The gaseous hydrocarbon feedstock is contacted with the catalyst as a mixture with an O₂-containing gas, preferably pure oxygen. The oxygen-containing gas may also comprise steam and/or CO₂ in addition to oxygen. Alternatively, the hydrocarbon feedstock is contacted with the catalyst as a mixture with a gas comprising steam and/or CO₂. For the purposes of this disclosure, the term “catalytic partial oxidation” or “net catalytic partial oxidation reaction” means that the CPOX reaction (Reaction (2)) predominates. However, other reactions such as steam reforming (see Reaction 1), dry reforming (Reaction (3)) and/or water-gas shift (Reaction (4)) may also occur to a lesser extent.

CH₄+CO₂←→2CO+2H₂ (3)

CO+H₂O←→CO₂+H₂ (4)

The relative amounts of the CO and H₂ in the reaction product mixture resulting from the net catalytic partial oxidation of the methane or natural gas and oxygen feed mixture are preferably about 2:1 H₂:CO, like the stoichiometric amounts of H₂ and CO produced in the partial oxidation reaction of Reaction (2).

As the preheated feed gas mixture passes over the catalyst to the point at which they ignite, an autothermal net catalytic partial oxidation reaction ensures. Preferably, the reaction conditions are maintained to promote continuation of the autothermal net catalytic partial oxidation process. For the purposes of this disclosure, “autothermal” means that after catalyst ignition, no additional heat must be supplied to the catalyst in order for the production of synthesis gas to continue. Autothermal reaction conditions are promoted by optimizing the concentrations of hydrocarbon and O₂ in the reactant gas mixture preferably within the range of about a 1.5:1 to about 2.3:1 ratio of carbon:oxygen. The hydrocarbon:oxygen ratio is the most important variable for maintaining the autothermal reaction and the desired product selectivities. Pressure, residence time, amount of feed preheat and amount of nitrogen dilution, if used, also affect the reaction products. All of these variables are preferably adjusted as necessary such that the desired H₂:CO ratio is achieved in the syngas emerging from the reactor. Preferably, the methane-containing feed and the oxygen-containing gas are mixed in such amounts to give a carbon (i.e., carbon in methane) to oxygen (i.e., molecular oxygen) ratio from about 1.5:1 to about 3.3:1, more preferably, from about 1.7:1 to about 2.1:1, and especially the stoichiometric ratio of 2:1. In some situations, such as when the methane-containing feed is a naturally occurring methane reserve, carbon dioxide may also be present in the methane-containing feed. It is expected that the CO₂ will not detrimentally affect the process. Depending on the particular situation, it may also be desirable at times to adjust the concentrations of the reactant gas mixture in order to increase or decrease the exothermicity of the process, maintain autothermal and enhance production of CO and H₂ at the desired ratio. The process is preferably operated at catalyst temperatures of from about 600° C. to about 2,000° C., preferably up to about 1,600° C. The hydrocarbon feedstock and the oxygen-containing gas are preferably pre-heated at a temperature between about 30° C. and about 750° C., more preferably not exceeding 500° C., before contact with the catalyst to facilitate light-off of the reaction. It is highly preferred that use of a supplemental burst of propane or other readily oxidizable gas added to the hydrocarbon stream is avoided.

The process may be operated at atmospheric or superatmospheric pressures, the latter being preferred. The pressures may be from about 100 kPa to about 32,000 kPa (about 1-320 atm), preferably from about 200 kPa to 10,000 kPa (about 2-100 atm). The hydrocarbon feedstock and the oxygen-containing gas may be passed over the catalyst at any of a variety of space velocities. Space velocities for the process, stated as gas hourly space velocity (GHSV), may be from about 20,000 to about 100,000,000 h⁻¹, preferably from about 100,000 to about 25,000,000 h⁻¹. Although for ease in comparison with prior art systems space velocities at standard conditions have been used to describe the present invention, it is well recognized in the art that residence time is the inversely related to space velocity and that the disclosure of high space velocities equates to low residence times on the catalyst. Under these operating conditions a flow rate of reactant gases is maintained sufficient to ensure a residence time of no more than 200 milliseconds with respect to each portion of reactant gas in contact with the catalyst. The product gas mixture emerging from the reactor is harvested and may be routed directly into any of a variety of applications.

One such application for the CO and H₂ product stream is for producing higher molecular weight hydrocarbon compounds using Fischer-Tropsch technology. Alternatively, the syngas product can serve as a source of H₂ (e.g., for fuel cells), in which case one of the above-described catalysts that provides enhanced selectivity for H₂ product may be selected, and process variables can be adjusted such that a H₂:CO ratio greater than 2:1 may be obtained, if desired. Fuel cells are chemical power sources in which electrical power is generated in a chemical reaction. The most common fuel cell is based on the chemical reaction between a reducing agent such as hydrogen and an oxidizing agent such as oxygen.

### EXAMPLE #1

### Preparation of Rh—Sm/Alumina Syngas Catalyst

0.4734 g Sm(NO₃)₃.5H₂O (Aldrich) was dissolved in sufficient water to form an aqueous solution. The alumina granules were immersed into the solution for wet impregnation, then allowed to dry on a hotplate. The impregnated granules were calcined in air according to the following schedule: 5° C./min ramp to 325° C., hold at 325° C. for 1 h, 5° C./min ramp to 700° C., hold at 700° C. for 2 h, cool down to room temperature. 0.5839 g RhCl₃.xH₂O (Aldrich) was dissolved in sufficient water to form an aqueous solution. The calcined Sm-containing granules were immersed into the rhodium solution for wet impregnation, then allowed to dry on a hotplate. The Rh impregnated granules were then calcined in air according to the following schedule: 5° C./min ramp to 325° C., hold at 325° C. for 1 h, 5° C./min ramp to 700° C., hold at 700° C. for 2 h, cool down to room temperature. This material was then reduced at 500° C. for 3 h under a stream of 300 mL/min H₂ and 300 mL/min N₂ to provide a catalyst containing 6% Rh and 5% Sm supported on ZrO₂ granules. Preferably the particles are no more than 3 mm in their longest characteristic dimension, or range from about 0.18 millimeter to about 3.2 millimeters. Preferably the support materials are pre-shaped as granules, spheres, pellets, or other geometry that provides satisfactory engineering performance, before application of the catalytic materials.

### EXAMPLE #2

### Light-off Catalyst Example #1 with Blank Floor

One gram of syngas catalyst prepared in Example #1 was tested following the test procedure with the bottom radiation shield being blank alumina monolith. No propane was spiked.

### EXAMPLE #3

### Light-off Catalyst Example #1 with Blank Floor and Propane Spike

Similar reactor loading was used as in Example #2. The reactor was lit-off following the standard test procedure with a propane spike.

### EXAMPLE #4

### Light-off Catalyst Example #1 with Pt on Alumina Monolith as Ignition Catalyst

The reactor was loaded the same way as was for Example #2, except that a Pt/alumina monolith was used as the bottom radiation shield.

### Preparation of Pt Ignition Catalyst

The Pt (1.23 wt %) alumina catalyst of this example was prepared by impregnation method. Suitable alumina monoliths about 10 or 15 mm long and 12 mm diameter are commercially available. Hydrogen hexachloroplatinate (IV) (H₂PtCl₆) (Aldrich), 8 wt % solution in water was used as platinum precursor. A platinum solution was made to have 1.45 wt % H₂PtCl₆ in water. Ten pieces of alumina monolith (½″ OD×⅝″ long; 80 ppi, A199 from Vesuvius) were soaked in the solution to saturate the pores (the porosity of the monolith was around 0.6 ml/g). The saturated alumina monoliths were then dried at 120° C. for 12 hours and calcined at 650° C. for 5 hours in flowing air at 50 ml/min. The resulting catalyst material contained 1.23 wt % platinum supported on the monolith.

### EXAMPLE #5

### Light-off Catalyst Example #1 with Pt Ce on Alumina Monolith as Ignition Catalyst

The reactor was loaded the same way as for Example #2, except that a Pt-Ce/alumina monolith was used as the bottom radiation shield.

The Pt-Ce alumina ignition catalyst of this example was prepared through the stepwise impregnation method. Hydrogen hexachloroplatinate (IV) (H₂PtCl₆) (Aldrich), 8 wt % solution in water was used as platinum precursor. Ce(NO₃)₃.6H₂O (Aldrich) was used as a precursor for CeO₂. First, Ce(NO₃)₃.6H₂O was dissolved into de-ionized water to obtain a cerium solution containing 8.13 wt % CeO₂. Then, ten pieces of the selected alumina monoliths (½″ OD×⅝″ long; 80 ppi, A199 from Vesuvius) was soaked in the solution to saturate the pores. The saturated monoliths were then dried at 120° C. for 12 hours and calcined at 400° C. for two hours. The monoliths were then soaked with aqueous platinum solution (containing 0.36 wt % H₂PtCl₆) to saturate the pore. The impregnated monoliths were then dried at 120° C. for 12 hours and calcined at 650° C. for 5 hours in flowing air at 50 ml/min. The resulting catalyst material contained 0.27 wt % Pt and 16.4 wt % CeO₂ on an alumina support.

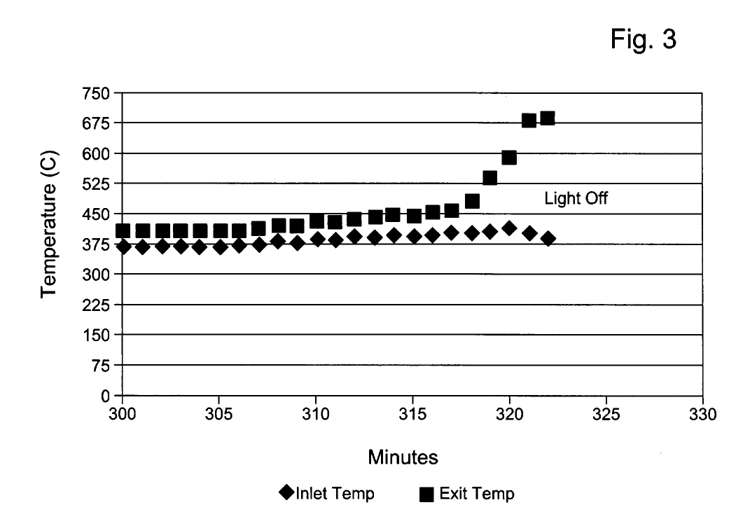

FIG. 3 illustrates the phenomena of rapid increase in exit temperature during light off. At approximately 310 minutes from the beginning of the process, the exit temperature suddenly increases from approximately 450° C. to 675° C.

Table 1 shows the results of testing Examples 2-5. Since there are unavoidable heat losses within the reactor system and the bed temperature was not directly measured. The ignition temperatures are identified by both the inlet and exit temperatures. The reactor performance is shown in Table 1 in the columns labeled CH₄ Conversion, H₂ Selectivity, and CO Selectivity. Identical syngas catalyst was used for Examples 2-5. In Example #2, the light-off did not occur after the reactor was heated up to as high as 435° C. and 453° C. at the inlet and outlet, respectively. When propane was added, as was in Example #3, the syngas reaction was initiated at inlet temperature of 404° C. and outlet temperature of 463° C. When the ignition catalyst was used downstream of the syngas catalyst, as was in Example #4 and #5; the ignition temperature was significantly decreased. The most striking result was demonstrated in Example #5, where the light-off occurred at an inlet temperature of 304° C. and outlet temperature of 312° C.

It should also be noted from Table 1 that for identical catalysts, the difference in performance (i.e., CH₄ Conversion, H₂ Selectivity, and CO Selectivity) is very small. Therefore, it is not expected that the addition of ignition catalyst will materially affect the performance of the selective oxidation catalyst.

Additionally, because there is no need for a propane feed, propane handling equipment is not necessary. This will result in a decrease in initial capital cost, and a decrease in operating costs during commercial scale-up.

TABLE 1
Experimental results on the effect of ignition catalyst.

| | | | Ignition | | | | |
|---|---|---|---|---|---|---|---|
| | | | Temperature | | Performance (%)* | | |
| Example | Ignition | Propane | (° C.) | | CH4 | H2 | CO |
| No. | catalyst | addition | Inlet | outlet | conversion | selectivity | selectivity |
| 2 | None | None | N/A | N/A | N/A | N/A | N/A |
| 3 | None | Yes | 404 | 463 | 91 | 90 | 95 |
| 4 | 1.23% Pt on | None | 370 | 422 | 92 | 90 | 95 |
| | alumina | | | | | | |
| 5 | 0.27% Pt/16.4% | None | 304 | 312 | 92 | 90 | 94 |
| | CeO2 on | | | | | | |
| | alumina | | | | | | |

*Reaction conditions: 1 gram catalyst as was prepared according to Example #1. Total gas flow rate = 5000 ml/min. Oxygen to methane molar ratio = 0.55. Pressure = atmospheric. Inlet temperature = 300° C.

It is envisioned that many igniters shapes and compositions would fall within the scope of the present invention. The igniter may be any shape as the situation would dictate. By way of example only, the igniter may be a monolithic disk, a packed bed of granules, a single point, or multiple points in the reactor. It is also envisioned that the igniter can be mixed or inserted into the reaction zone.

It is also envisioned that in some instances it may be beneficial to have an intermediate material between the igniter and the reaction zone. For example, it may be desirable to insert an intermediate material (such as an inert refractory material or some other kind of catalyst) if the ignition agent reacts adversely with the catalyst in the reaction zone or if it is desirable to conduct a secondary reaction behind the primary reaction and above the ignition reaction.

Should the disclosure of any of the patents and publications that are incorporated herein by reference conflict with the present specification to the extent that it might render a term unclear, the present specification shall take precedence.

While the preferred embodiments of the invention have been disclosed herein, it will be understood that various modifications can be made to the system described herein without departing from the scope of the invention. Without further elaboration, it is believed that one skilled in the art can, using the description herein, utilize the present invention to its fullest extent.

What is claimed is:

1. A process for start-up of a thermally self-sustaining reaction, the process comprising:
introducing a reactant stream to a reactor, wherein the reactor comprises:
a reaction zone for conducting the thermally self-sustaining reaction; and
an ignition zone in thermal contact with the reaction zone;
wherein the at least a portion of the ignition zone is downstream of at least a portion of the reaction zone;
wherein the ignition zone comprises an igniter;
wherein the igniter ignites the reactant stream and starts-up the thermally self-sustaining reaction in the reaction zone.

2. The process of claim 1 wherein the thermally self-sustaining reaction is a partial oxidation reaction.

3. The process of claim 2 wherein the reactant stream comprises a hydrocarbon-containing gas and an oxygen-containing gas.

4. The process of claim 1 wherein the self-sustaining reaction is an oxidative dehydrogenation reaction.

5. The process of claim 1 wherein the self-sustaining reaction is a sulfur partial oxidation reaction.

6. The process of claim 1 wherein the reaction zone comprises a catalyst.

7. The process of claim 6 wherein the catalyst material comprises at least one of nickel, ruthenium, palladium, osmium, iridium, samarium, cobalt, platinum, rhodium, Ni—MgO, Group VIII metals, or combinations thereof.

8. The process of claim 6 wherein the catalyst material comprises rhodium supported on alumina or zirconia granules having a diameter between about 0.18 mm and about 3 mm.

9. The process of claim 8 wherein the catalyst material further comprises samarium.

10. The process of claim 1 wherein the ignition zone physically contacts the reaction zone.

11. The process of claim 1 wherein the ignition zone comprises Rh, Ru, Pd, Pt, Au, Ag, Os, Ir, Sc, Ti, V, Cr, Mn, Fe, Co, Ni, Cu, Zn, Nb, Mo, Tc, Ru, Rh, Pd, Ag, Hf, Ta, W, or Re.

12. The process of claim 1 wherein the igniter comprises platinum.

13. The process of claim 12 wherein the igniter further comprises an element selected from the group consisting of Mg, Al, Ti, Zr and Si.

14. The process of claim 12 wherein the igniter further comprises cerium.

15. The process of claim 1 wherein the igniter comprises cerium.

16. The process of claim 1 wherein the reactor further comprises a second reaction zone downstream of the ignition zone.

17. The process of claim 1 wherein a downstream portion of the reaction zone and an upstream portion of the ignition zone commingle.

18. A process for the production of liquid hydrocarbons, the process comprising:
introducing a stream comprising a hydrocarbon-containing gas and an oxygen-containing gas to a reactor, wherein the reactor comprises:
a reaction zone; and
an ignition zone comprising an igniter;
wherein at least a portion of the ignition zone is disposed downstream of and in thermal contact with the reaction zone;
igniting the stream so as to initiate the partial oxidation reaction in the reaction zone to produce a product stream comprising synthesis gas;
contacting at least a portion of the product stream with a catalyst in a hydrocarbon synthesis reactor so as to convert at least a portion of the synthesis gas to liquid hydrocarbons.

19. The process of claim 18 wherein the hydrocarbon-containing gas comprises natural gas.

20. The process of claim 18 wherein the reaction zone comprises a catalyst material.

21. The process of claim 20 wherein the catalyst material comprises at least one of nickel, ruthenium, palladium, osmium, iridium, samarium, cobalt, platinum, rhodium, Ni—MgO, Group VIII metals, or combinations thereof.

22. The process of claim 20 wherein the catalyst material comprises rhodium supported on alumina or zirconia granules having a diameter between about 0.18 mm and about 3 mm.

23. The process of claim 22 wherein the catalyst material further comprises samarium.

24. The process of claim 18 wherein the oxygen-containing gas comprises substantially pure oxygen.

25. The process of claim 18 wherein the ignition zone physically contacts the reaction zone.

26. The process of claim 18 wherein a downstream portion of the reaction zone and an upstream portion of the ignition zone commingle.

27. The process of claim 18 wherein the ignition zone comprises Rh, Ru, Pd, Pt, Au, Ag, Os, Ir, Sc, Ti, V, Cr, Mn, Fe, Co, Ni, Cu, Zn, Nb, Mo, Tc, Ru, Rh, Pd, Ag, Hf, Ta, W, or Re.

28. The process of claim 18 wherein the igniter comprises platinum.

29. The process of claim 28 wherein the igniter further comprises cerium.

30. The process of claim 18 wherein the igniter comprises cerium.

31. The process of claim 18 wherein the reactor further comprises a second reaction zone, at least a portion of which is downstream of the reaction zone.

32. A reactor for producing synthesis gas from a feedstream comprising a hydrocarbon-containing gas and an oxygen-containing gas, the reactor comprising:
a reaction zone for partially oxidizing the hydrocarbon gas to a product stream comprising synthesis gas; and
an igniter in thermal contact with the reaction zone, wherein at least a portion of the igniter is downstream of the reaction zone and wherein the igniter ignites the hydrocarbon in the reaction zone so as to start-up the partial oxidation reaction in the reaction zone.

33. The reactor of claim 32 wherein the hydrocarbon-containing gas comprises primarily natural gas.

34. The reactor of claim 32 wherein the reaction zone comprises a catalytic material for catalyzing the partial oxidation reaction in the reaction zone.

35. The reactor of claim 34 wherein the catalytic material comprises rhodium supported on alumina or zirconia granules having a diameter between about 0.18 mm and about 3 mm.

36. The reactor of claim 35 wherein the catalytic material further comprises samarium.

37. The process of claim 34 wherein the catalytic material comprises at least one of nickel, ruthenium, palladium, osmium, iridium, samarium, cobalt, platinum, rhodium, Ni—MgO, Group VIII metals, or combinations thereof.

38. The-process of claim 32 wherein the igniter is in physical contact with the reaction zone.

39. The process of claim 32 wherein the ignition zone comprises Rh, Ru, Pd, Pt, Au, Ag, Os, Ir, Sc, Ti, V, Cr, Mn, Fe, Co, Ni, Cu, Zn, Nb, Mo, Tc, Ru, Rh, Pd, Ag, Hf, Ta, W, or Re.

40. The process of claim 32 wherein the igniter comprises platinum.

41. The process of claim 40 wherein the igniter further comprises cerium.

42. The process of claim 32 wherein the igniter comprises cerium.

43. The process of claim 32 wherein a downstream portion of the reaction zone is commingled with an upstream portion of the igniter.

44. A means for initiating a thermally self-sustaining reaction, the means comprising:
a means for reacting a reactant stream, wherein the means for reacting comprises:
a reaction zone for conducting the thermally self-sustaining reaction; and
means for igniting in thermal contact with the reaction zone;
wherein the at least a portion of the means for igniting is downstream of at least a portion of the reaction zone.
